(19) United States
(12) Patent Application Publication
(10) Pub. No.: US 2023/0078303 A1
(43) Pub. Date: Mar. 16, 2023

(54) SYSTEMS AND METHODS OF HEALTH SCREENING VEHICLE PASSENGERS

(71) Applicant: Honda Motor Co., Ltd., Tokyo (JP)

(72) Inventor: Jerry Erin Bonnah, Huntington Beach, CA (US)

(21) Appl. No.: 17/476,945

(22) Filed: Sep. 16, 2021

(51) Int. Cl. A61B 5/00; A61B 5/01; B60W 40/08; B60W 50/14; B60W 50/10

(52) U.S. Cl. CPC A61B 5/0008; A61B 5/01; B60W 40/08; B60W 50/14; B60W 50/10; A61B 2562/0271; B60W 2040/0872; B60W 2540/221; B60W 2050/146; B60W 2510/00

(57) ABSTRACT

A system for use in health screening passengers of a vehicle. The system includes a temperature sensor within a passenger compartment of the vehicle. The temperature sensor is configured to determine a body temperature of the at least one passenger within the passenger compartment. The system also includes a transmitter configured to wirelessly transmit body temperature data determined by the temperature sensor, and a server. The server includes an input for receiving communications from the transmitter, an output for communicating with a client device, and a processor in communication with a memory. The processor receives, from the vehicle via the input, body temperature data associated with at least one passenger within the vehicle, analyzes the body temperature data relative to a body temperature threshold, wherein the body temperature threshold is associated with an elevated body temperature, and outputs, to the client device via the output, a result of the analysis.

### BACKGROUND

The present disclosure relates generally to health screening and, more specifically, to systems and methods of processing a vehicle passenger's body temperature data for use in preemptive health screening.

Recent global health crises have forced the population at large to be acutely aware of the health of those around them. For example, the most recent crisis has generated an increased emphasis on mask wearing, physical distancing, and monitoring close contacts for potential symptoms of illness. The most recent crisis has also significantly altered people's lifestyles as a whole, including relying on curbside pickup for essential goods, increasing use of delivery services, and taking enhanced precautions when using ridesharing services. One symptom known to be correlated with illness is elevated body temperature, which can be determined by contact and non-contact type thermometers. At least some known non-contact type thermometers are even used to measure the body temperature of individuals as they enter a building, for example. However, such health screening procedures only screen individuals on site, which may be ineffective at screening ill individuals before they can come into close contact with others in the community.

### BRIEF DESCRIPTION

In one aspect, a system for use in health screening passengers of a vehicle is provided. The system includes a first temperature sensor within a passenger compartment of the vehicle. The first temperature sensor is configured to determine a body temperature of the at least one passenger within the passenger compartment. The system also includes a transmitter configured to wirelessly transmit body temperature data determined by the first temperature sensor, and a server. The server includes an input for receiving communications from the transmitter, an output for communicating with a client device, and a processor in communication with a memory. The processor is programmed to receive, from the vehicle via the input, body temperature data associated with at least one passenger within the vehicle, analyze the body temperature data relative to a body temperature threshold, wherein the body temperature threshold is associated with an elevated body temperature, and output, to the client device via the output, a result of the analysis.

In another aspect, a vehicle is provided. The vehicle includes a passenger compartment and a first temperature sensor within the passenger compartment. The first temperature sensor is configured to determine a body temperature of at least one passenger within the passenger compartment. The vehicle also includes a transmitter configured to wirelessly transmit body temperature data determined by the first temperature sensor.

In yet another aspect, a system for use in health screening passengers of a vehicle is provided. The system includes an input for communicating with the vehicle, an output for communicating with a client device, and a processor in communication with a memory. The processor is programmed to receive, from the vehicle via the input, body temperature data associated with at least one passenger within the vehicle, analyze the body temperature data relative to a body temperature threshold, wherein the body temperature threshold is associated with an elevated body temperature, and output, to the client device via the output, a result of the analysis.

### BRIEF DESCRIPTION OF THE DRAWINGS

FIG. 1 is a perspective view of an exemplary vehicle.

FIG. 2 is a driver seat view from within a passenger compartment of the vehicle shown in FIG. 1.

FIG. 3 is a side interior view of the passenger compartment of the vehicle shown in FIG. 1.

FIG. 4 is a block diagram illustrating an exemplary health screening system that may include the vehicle shown in FIG. 1.

### DETAILED DESCRIPTION

The embodiments described herein relate generally to systems and methods of processing a vehicle passenger's body temperature data for use in preemptive health screening. The systems include a vehicle, such as an automobile, fitted with a temperature sensor in the passenger compartment for determining the body temperature of passengers within the vehicle. The body temperature data is transmitted to and stored within a server, and the body temperature data is analyzed to determine if any of the passengers have an elevated body temperature, which is an early warning sign of potential illness. The server is in communication with a client device owned and operated by a third party. The client device may periodically request a result of the body temperature analysis. For example, an individual may order goods from the third party and may elect to receive the goods via curbside pickup, in which an employee of the third party must approach the individual to provide them with the goods. In such an example, the individual uses the vehicle described herein to reach a retail location of the third party, and the body temperature of the individual may be analyzed en route or upon arrival at the retail location. The result of the analysis may be provided to the third party before any interaction between the vehicle passengers and the employee occurs. Accordingly, the employee is forewarned and protected from exposure to potentially ill individuals, thereby reducing the spread of infectious diseases.

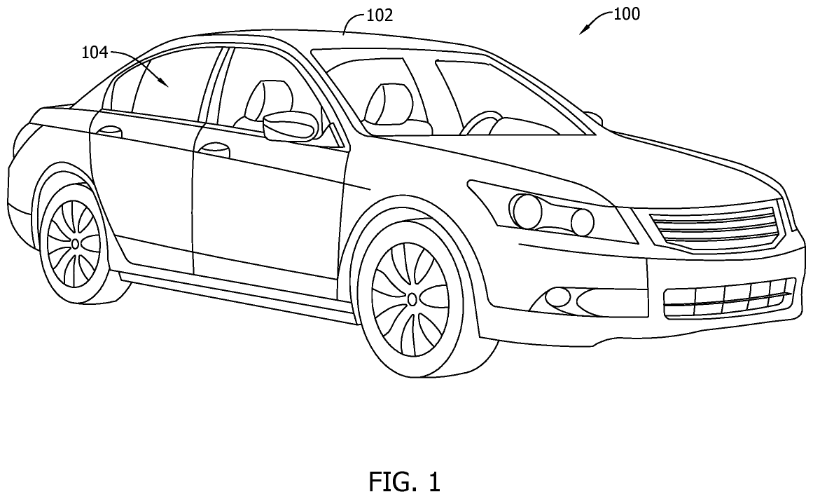

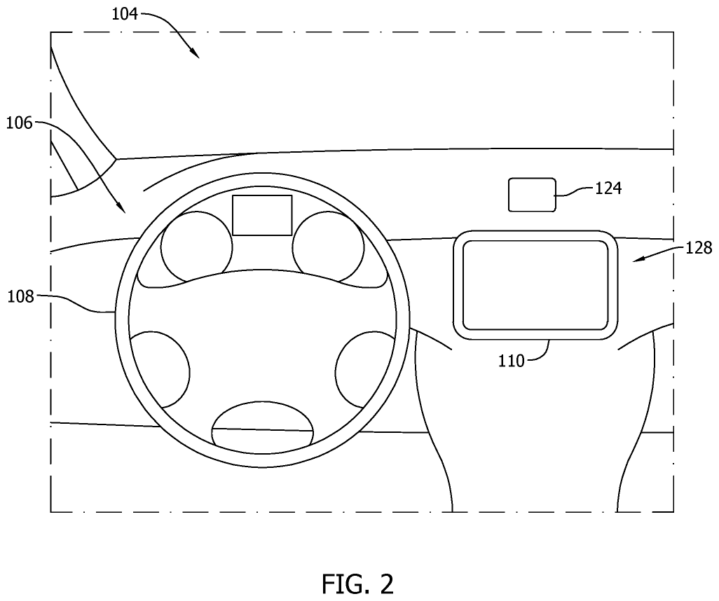

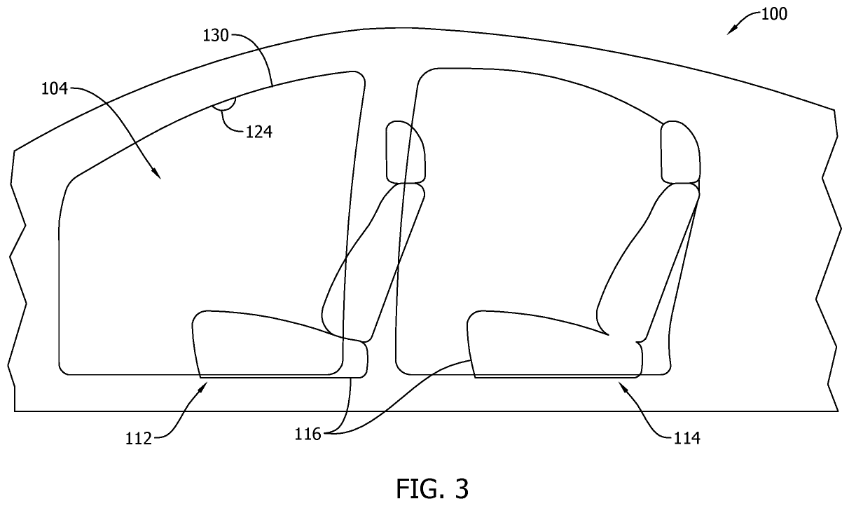

FIGS. 1-3 illustrate various views of an exemplary vehicle 100, such as a personal automobile. Vehicle 100 includes a body structure 102 that defines a passenger compartment 104 of vehicle 100. Referring to FIG. 2, passenger compartment 104 includes a driver's position 106, in which a wheel 108 and an interior display 110 are accessible to the driver (not shown) of vehicle 100. Referring to FIG. 3, passenger compartment 104 includes a first row 112 and a second row 114 of passenger seats 116. Accordingly, passengers (not shown) of vehicle 100 may be seated in first and/or second rows 112 and 114.

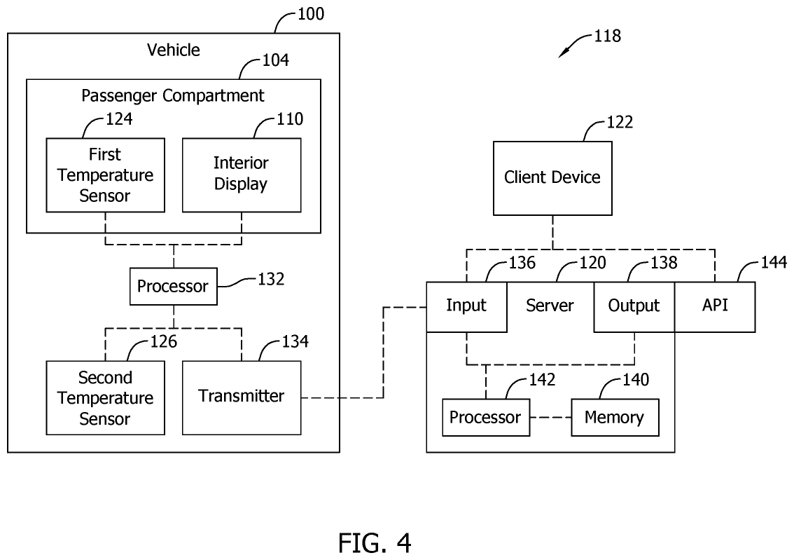

FIG. 4 is a block diagram illustrating an exemplary health screening system 118 that may include vehicle 100. In the exemplary embodiment, system 118 includes vehicle 100 and a server 120 communicatively coupled with a client device 122. Vehicle 100 includes a first temperature sensor 124 within passenger compartment 104, and a second temperature sensor 126. Second temperature sensor 126 is configured to determine an ambient temperature exterior of vehicle 100. First temperature sensor 124 is configured to determine a body temperature of passengers within vehicle 100.

First temperature sensor 124 is any contact or non-contact type thermometer that enables system 118 to function as described herein. For example, first temperature sensor 124 may be a non-contact type infrared scanner capable of determining the body temperatures of one or more passengers of vehicle 100 within its field of view simultaneously. Accordingly, first temperature sensor 124 is positioned within passenger compartment 104 to provide line of sight between first temperature sensor 124 and each designated passenger seat 116 within passenger compartment 104. For example, referring to FIG. 2 and in one embodiment, first temperature sensor 124 is positioned on a center console 128 within passenger compartment 104. Accordingly, first temperature sensor 124 has line of sight to first row 112 of passenger seats 116, and has line of sight to second row 114 through the gap defined between passenger seats 116 of first row 112. Alternatively, referring to FIG. 3, first temperature sensor 124 is coupled to a headliner 130 of passenger compartment 104. Thus, first temperature sensor 124 is positioned to analyze exposed skin of all passengers of vehicle 100 from a fixed position within passenger compartment 104. In an alternative embodiment, vehicle 100 may have more than one first temperature sensor 124 to enable the body temperature of each passenger within vehicle 100 to be determined.

In operation, in one embodiment, first temperature sensor 124 initiates a body temperature scan based on a manual input received from a passenger within vehicle 100. For example, the manual input may be received at interior display 110, which is used to enable passengers to control various functions of vehicle 100. In another embodiment, first temperature sensor 124 automatically initiates the body temperature scan when one or more criteria are met. For example, the body temperature scan may be initiated based on a change in an operational status of vehicle 100 that is monitored by a processor 132 of vehicle 100. Processor 132 may be operable to monitor at least one of opening or closing a door of the vehicle, starting or shutting off of the vehicle, a location of the vehicle relative to a destination, or a transmission of the vehicle to determine the change in the operational status. Such changes in the operational status are generally indicative of vehicle occupancy. Thus, monitoring these changes in the operational status of vehicle 100 enables first temperature sensor 124 to be passively active, thereby conserving the energy of vehicle 100.

In some embodiments, it is beneficial to initiate a body temperature scan as vehicle 100 reaches its destination and there is a greater likelihood of interaction between the vehicle passengers and third party individuals. For example, shutting vehicle 100 off and/or switching the transmission from a drive mode to a park mode may be indicative of vehicle 100 reaching its destination. Accordingly, initiating body temperature scans based on these monitored changes in the vehicle's operational status enables the most current, and thus most relevant, body temperature readings of the vehicle passengers to be obtained.

It may also be beneficial to initiate the body temperature scan on a delay after a change in operational status is detected. As described above, second temperature sensor 126 monitors an ambient temperature exterior of vehicle 100. Both comparatively low and high ambient temperatures, relative to a normal body temperature threshold, may result in inaccurate non-contact body temperature measurements being obtained based on the temporary effect ambient temperatures have on the temperature of an individual's skin. In one embodiment, processor 132 compares the current ambient temperature to an ambient temperature threshold range. If processor 132 determines the current ambient temperature is outside the ambient temperature threshold range, processor 132 directs first temperature sensor 124 to initiate the body temperature scan on a delay (e.g., one minute, two minutes, or five minutes) to allow the vehicle passenger's skin temperature to normalize, thereby enabling a more accurate determination of their body temperature to be made.

In one embodiment, processor 132 analyzes the body temperature(s) of passenger(s) within vehicle 100 relative to a body temperature threshold, and outputs a result of the analysis to interior display 110 for viewing from within passenger compartment 104. The body temperature threshold is associated with an elevated body temperature that may be indicative of potential illness. The body temperature threshold may be 100° F., 100.4° F., or any other threshold deemed appropriate by regulatory health agencies. In an alternative embodiment, vehicle 100 includes a transmitter 134 for wirelessly transmitting body temperature data determined by first temperature sensor 124 to server 120.

Server 120 includes an input 136 for communicating with vehicle 100 and client device 122, and an output 138 for communicating with client device 122. Server 120 also includes a memory 140 and a processor 142, including hardware and software, coupled to memory 140 for executing programmed instructions. Processor 142 may include one or more processing units (e.g., in a multi-core configuration) and/or include a cryptographic accelerator (not shown). Server 120 is programmable to perform one or more operations described herein by programming memory 140 and/or processor 142. For example, processor 142 may be programmed by encoding an operation as executable instructions and providing the executable instructions in memory 140.

Processor 142 may include, but is not limited to, a general purpose central processing unit (CPU), a microcontroller, a microprocessor, a reduced instruction set computer (RISC) processor, an open media application platform (OMAP), an application specific integrated circuit (ASIC), a programmable logic circuit (PLC), and/or any other circuit or processor capable of executing the functions described herein. The methods described herein may be encoded as executable instructions embodied in a computer-readable medium including, without limitation, a storage device and/or a memory device. Such instructions, when executed by processor 142, cause processor 142 to perform at least a portion of the functions described herein. The above examples are for example purposes only, and thus are not intended to limit in any way the definition and/or meaning of the term processor.

Memory 140 is one or more devices that enable information such as executable instructions and/or other data to be stored and retrieved. Memory 140 may include one or more computer-readable media, such as, without limitation, dynamic random access memory (DRAM), synchronous dynamic random access memory (SDRAM), static random access memory (SRAM), a solid state disk, and/or a hard disk. Memory 140 may be configured to store, without limitation, executable instructions, operating systems, applications, resources, installation scripts and/or any other type of data suitable for use with the methods and systems described herein.

Instructions for operating systems and applications are located in a functional form on non-transitory memory 140 for execution by processor 142 to perform one or more of the processes described herein. These instructions in the different implementations may be embodied on different physical or tangible computer-readable media, such as memory 140 or another memory, such as a computer-readable media (not shown), which may include, without limitation, a flash drive and/or thumb drive. Further, instructions may be located in a functional form on non-transitory computer-readable media, which may include, without limitation, smart-media (SM) memory, compact flash (CF) memory, secure digital (SD) memory, memory stick (MS) memory, multimedia card (MMC) memory, embedded-multimedia card (e-MMC), and micro-drive memory. The computer-readable media may be selectively insertable and/or removable from server 120 to permit access and/or execution by processor 142. In an alternative implementation, the computer-readable media is not removable.

Processor 142 is programmed to receive, from vehicle 100 via input 136, body temperature data associated with one or more passengers within vehicle 100. The body temperature data may be stored in memory 140 before being analyzed. Processor 142 analyzes the body temperature data relative to the body temperature threshold, similar to the process described above with respect to processor 132. Processor 142 may then output, to client device 122 via output 138, a result of the analysis. In an alternative embodiment, processor 142 outputs the body temperature data to client device 122, and client device 122 performs its own analysis.

In one embodiment, processor 142 outputs the result of the analysis, or the body temperature data, only after receiving a request from client device 122. Client device 122 may be associated with any commercial entity or third party in which interaction or close contact with passengers of vehicle 100 may occur. Example commercial entities include, but are not limited to including, a ridesharing service, a retail merchant, and a restaurant. Accordingly, in the exemplary embodiment, processor 142 outputs the result of the analysis to client device 122 via an application programming interface (API) 144. A corresponding API (not shown) on client device 122 may be used to display the result of the analysis. In the event the result of the analysis indicates one or more passengers of vehicle 100 has an elevated body temperature, a user of client device 122 may change their behavior or take additional precautions to reduce close contact with the passengers. For example, in a curbside delivery scenario, the user of client device 122 may elect to deliver the passenger's purchased goods to the trunk of vehicle 100, rather than interact directly with the passenger.

The embodiments described herein relate to a health screening system for use in providing early detection of potential illness in passengers of a vehicle. The system includes a temperature sensor within the vehicle, and body temperature data determined by the sensor are transmitted to a central database for analysis and distribution to third party entities. The result of the analysis may be utilized by the third party entities to be forewarned of potential health hazards, and the third party entities may elect to take appropriate measures for protecting employees thereof from the hazards. Accordingly, the system described herein facilitates reducing the spread of infectious diseases.

Exemplary embodiments of a health screening system are described above in detail. Although the health screening system is described and illustrated in association with a automobiles, the invention is also intended for use with any passenger carrying vehicle as well. Moreover, it should also be noted that the components of the invention are not limited to the specific embodiments described herein, but rather, aspects of each component may be utilized independently and separately from other components and methods described herein.

This written description uses examples to disclose various embodiments, including the best mode, and also to enable any person skilled in the art to practice the various implementations, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for use in health screening passengers of a vehicle, the system comprising:
a first temperature sensor within a passenger compartment of the vehicle, the first temperature sensor configured to determine a body temperature of the at least one passenger within the passenger compartment;
a transmitter configured to wirelessly transmit body temperature data determined by the first temperature sensor; and
a server comprising:
an input for receiving communications from the transmitter;
an output for communicating with a client device; and
a processor in communication with a memory, the processor programmed to:
receive, from the vehicle via the input, body temperature data associated with at least one passenger within the vehicle;
analyze the body temperature data relative to a body temperature threshold, wherein the body temperature threshold is associated with an elevated body temperature; and
output, to the client device via the output, a result of the analysis.

2. The system in accordance with claim 1, wherein the first temperature sensor is configured to automatically initiate a body temperature scan of the at least one passenger based on a change in an operational status of the vehicle.

3. The system in accordance with claim 2 further comprising a processor configured to monitor at least one of opening or closing a door of the vehicle, starting or shutting off of the vehicle, a location of the vehicle relative to a destination, or a transmission of the vehicle to determine the change in the operational status.

4. The system in accordance with claim 1, wherein the first temperature sensor is configured to determine a body temperature of the at least one passenger at least one of en route or upon arrival at destination of the vehicle.

5. The system in accordance with claim 1, wherein the processor is further configured to output the result of the analysis to the client device via an application programming interface (API).

6. The system in accordance with claim 1, wherein the processor is further configured to receive, from the client device, a request for the result of the analysis before outputting the result of the analysis to the client device.

7. A vehicle comprising:
a passenger compartment;
a first temperature sensor within the passenger compartment, the first temperature sensor configured to determine a body temperature of at least one passenger within the passenger compartment; and
a transmitter configured to wirelessly transmit body temperature data determined by the first temperature sensor.

8. The vehicle in accordance with claim 7 further comprising a processor configured to:
analyze the body temperature of the at least one passenger relative to a body temperature threshold; and
output a result of the analysis.

9. The vehicle in accordance with claim 8 further comprising an interior display within the passenger compartment, wherein the interior display is configured to display the result of the analysis.

10. The vehicle in accordance with claim 9, wherein the first temperature sensor is configured to initiate a body temperature scan of the at least one passenger based on a manual input received at the interior display.

11. The vehicle in accordance with claim 8, wherein the processor outputs the result of the analysis before any interaction between a passenger of the vehicle and a third party individual occurs.

12. The vehicle in accordance with claim 7, wherein the first temperature sensor is configured to automatically initiate a body temperature scan of the at least one passenger based on a change in an operational status of the vehicle.

13. The vehicle in accordance with claim 12 further comprising a processor configured to monitor at least one of opening or closing a door of the vehicle, starting or shutting off of the vehicle, a location of the vehicle relative to a destination, or a transmission of the vehicle to determine the change in the operational status.

14. The vehicle in accordance with claim 12, wherein the first temperature sensor is configured to initiate the body temperature scan on a delay after the change in the operational status is detected.

15. The vehicle in accordance with claim 14 further comprising a second temperature sensor configured to determine an ambient temperature external of the vehicle, wherein the first temperature sensor is configured to initiate the body temperature scan on the delay if the ambient temperature is outside of an ambient temperature threshold range.

16. A system for use in health screening passengers of a vehicle, the system comprising:
an input for communicating with the vehicle;
an output for communicating with a client device;
a processor in communication with a memory, the processor programmed to:
receive, from the vehicle via the input, body temperature data associated with at least one passenger within the vehicle;
analyze the body temperature data relative to a body temperature threshold, wherein the body temperature threshold is associated with an elevated body temperature; and
output, to the client device via the output, a result of the analysis.

17. The system in accordance with claim 16, wherein the processor is further configured to output the result of the analysis to the client device via an application programming interface (API).

18. The system in accordance with claim 16, wherein the processor is further configured to receive, from the client device, a request for the result of the analysis before outputting the result of the analysis to the client device.

19. The system in accordance with claim 16, wherein the processor is further configured to:
store the body temperature data in the memory; and
retrieve and analyze the body temperature data upon receiving a request from the client device.

20. The system in accordance with claim 16 further comprising the vehicle that comprises:
a passenger compartment;
a first temperature sensor within the passenger compartment, the first temperature sensor configured to determine a body temperature of the at least one passenger within the passenger compartment; and
a transmitter configured to wirelessly transmit the body temperature data, determined by the first temperature sensor, to the input.
